(19) United States
(12) Patent Application Publication
(10) Pub. No.: US 2003/0229237 A1
(43) Pub. Date: Dec. 11, 2003

(54) In situ production of fatty acid alkyl esters

(76) Inventors: MIchael J. Haas, Oreland, PA (US); Thomas A. Foglia, Lafayette Hill, PA (US)

Correspondence Address: USDA, ARS, OTT, 5601 SUNNYSIDE AVE, RM 4-1159, BELTSVILLE, MD, 20705-5131, US

(21) Appl. No.: 10/404,409

(22) Filed: Apr. 1, 2003

Related U.S. Application Data
(60) Provisional application No. 60/369,370, filed on Apr. 2, 2002.

(51) Int. Cl. C07C 51/43

(52) U.S. Cl. 554/174

(57) ABSTRACT

The present invention relates to a method for producing fatty acid alkyl esters, involving transesterifying a feedstock containing lipid-linked fatty acids with an alcohol and an alkaline catalyst to form fatty acid alkyl esters, wherein the feedstock is selected from soy, coconut, corn, cotton, flax, palm, rapeseed/canola, safflower, sunflower, animal fats and oils, or mixtures thereof, and wherein the feedstock has not been treated (e.g., extraction with an organic solvent or by extruder/expeller technology) to release the lipid components of the feedstock.

### REFERENCE TO RELATED APPLICATION

This application claims the benefit of U.S. Provisional Application No. 60/369,370, filed Apr. 2, 2002, which is incorporated herein by reference in its entirety.

### BACKGROUND OF THE INVENTION

The present invention relates to a method for producing fatty acid alkyl esters, involving transesterifying a feedstock containing lipid-linked fatty acids with an alcohol and an alkaline catalyst to form fatty acid alkyl esters, wherein the feedstock is selected from soy, coconut, corn, cotton, flax, palm, rapeseed/canola, safflower, sunflower, animal fats and oils, or mixtures thereof, and wherein the feedstock has not been treated to release the lipid components of the feedstock.

Over the past three decades interest in the reduction of air pollution, and in the development of domestic energy sources, has triggered research in many countries on the development of non-petroleum fuels for internal combustion engines. For compression ignition (diesel) engines, it has been shown that the simple alcohol esters of fatty acids (biodiesel) are acceptable alternative diesel fuels. Biodiesel has a higher oxygen content than petroleum diesel, and therefore reduces emissions of particulate matter, hydrocarbons, and carbon monoxide, while also reducing sulfur emissions due to a low sulfur content (Sheehan, J., et al., Life Cycle Inventory of Biodiesel and Petroleum Diesel for Use in an Urban Bus, National Renewable Energy Laboratory, Report NREL/SR-580-24089, Golden, Colo. (1998); Graboski, M. S., and R. L. McCormick, Prog. Energy Combust. Sci., 24:125-164 (1998)). Since it is made from agricultural materials, which are produced via photosynthetic carbon fixation (e.g., by plants and by animals that consume plants), the combustion of biodiesel does not contribute to net atmospheric carbon levels.

Initial efforts at the production, testing, and use of biodiesel employed refined edible vegetable oils (expelled or recovered by solvent extraction of oilseeds) and animal fats (e.g., beef tallow) as feedstocks for fuel synthesis (Krawczyk, T., INFORM, 7: 800-815 (1996); Peterson, C. L., et al., Applied Engineering in Agriculture, 13: 71-79 (1997); Holmberg, W. C., and J. E. Peeples, Biodiesel: A Technology, Performance, and Regulatory Overview, National SoyDiesel Development Board, Jefferson City, Mo. (1994); Freedman, B., et al., J. Am. Oil Chem. Soc., 61(10): 1638-1643 (1984)). More recently, methods have been developed to produce fatty acid methyl esters (FAME) from cheaper, less highly refined lipid feedstocks such as spent restaurant grease and soybean soapstock (Mittelbach, M., and P. Tritthart, J. Am Oil Chem. Soc., 65(7):1185-1187 (1988); Graboski, M. S., et al., The Effect of Biodiesel Composition on Engine Emissions from a DDC Series 60 Diesel Engine, Final Report to USDOE/National Renewable Energy Laboratory, Contract No. ACG-8-17106-02 (2000); Haas, M. J., et al., Enzymatic Approaches to the Production of Biodiesel Fuels, in Kuo, T. M. and Gardner, H. W. (Eds.), Lipid Biotechnology, Marcel Dekker, Inc., New York. (2002); Canakci, M., and J. Van Gerpen, Biodiesel Production from Oils and Fats with High Free Fatty Acids, Abstracts of the 92^{nd} American Oil Chemists' Society Annual Meeting & Expo, p. S74 (2001); U.S. Pat. Nos. 2,383,601; 2,494,366; 4,695,411; 4,698,186; 4,164,506; Haas, M. J., et al., J. Am. Oil Chem. Soc., 77:373-379 (2000); Haas, M. J., et al., Energy & Fuels, 15(5):1207-1212 (2001)).

We now report the production of fatty acid alkyl esters using as substrate unextracted lipids still residing in the agricultural materials in which they were produced. Our method achieved the desired transesterification of the lipid-linked fatty acids by direct treatment of the lipid source itself with alcohol and an alkaline catalyst. Because no prior isolation or purification of the lipid in the lipid source is involved, this method for ester synthesis should have a greatly reduced cost compared to existing methods since it eliminates the need for costly expelling/extraction and refining steps currently employed to produce the fats and oils that are the feedstock for fatty acid ester synthesis.

### SUMMARY OF THE INVENTION

The present invention relates to a method for producing fatty acid alkyl esters, involving transesterifying a feedstock containing lipid-linked fatty acids with an alcohol and an alkaline catalyst to form fatty acid alkyl esters, wherein the feedstock is selected from soy, coconut, corn, cotton, flax, palm, rapeseed/canola, safflower, sunflower, animal fats and oils, or mixtures thereof, and wherein the feedstock has not been treated to release the lipid components of the feedstock.

### BRIEF DESCRIPTION OF THE DRAWINGS

FIGS. 1-4 show the results obtained upon analysis by thin layer chromatography of the products obtained when soy flakes were subjected, under various conditions, to the process described herein;

FIG. 5 shows predicted response surfaces, calculated from Eqn. 1-3 below, for the product composition after 2 hours of in situ transesterification of 5.00 g of soy flakes at 60° C., as a function of the amount of alcohol and the concentration of sodium hydroxide; (A) FAME; (B) TAG; (C) FFA;

FIG. 6 shows predicted response surfaces, calculated from Eqn. 1-3 below, for the product composition after 6 hours in situ transesterification of 5.00 g of soy flakes at 60° C., as a function of the amount of alcohol and the concentration of sodium hydroxide; (A) FAME; (B) TAG; (C) FFA;

FIG. 7 shows predicted response surfaces, calculated from Eqn. 3 and 4 below, for the product composition after 8 h of in situ transesterification of 5.00 g soy flakes at 23° C., as a function of the amount of alcohol and the concentration of sodium hydroxide; (A) FAME; (B) FFA.

### DETAILED DESCRIPTION OF THE INVENTION

The present invention relates to a method for producing fatty acid alkyl esters, involving transesterifying a feedstock containing lipid-linked fatty acids with an alcohol and an alkaline catalyst to form fatty acid alkyl esters, wherein the feedstock is selected from soy, coconut, corn, cotton, flax, palm, rapeseed/canola, safflower, sunflower, animal fats and oils, or mixtures thereof, and wherein the feedstock has not been treated to release the lipid components of the feedstock.

The process described herein is not feedstock-limited and is expected to achieve highly efficient fatty acid alkyl ester (e.g., fatty acid methyl ester) synthesis using materials that usually provide a source of plant oil, including, but not limited to, soy, coconut, corn, cotton, flax, palm, rapeseed/canola, safflower, and sunflower seeds or fruits. The process may also use unrefined animal fat, such as tallow, lard, fish oil, or similar material. Generally, the feedstocks may need some pretreatment in order to expose a greater surface area (e.g, such as flaking in the case of soybeans) which will greatly improve the efficiency and reduce the time-to-completion of the process. A preferred feedstock for the process of the present invention in the United States is soybeans because soybeans are the predominant oilseed processed in the United States. Generally the lipid source (e.g., oilseeds) is flaked or ground to provide additional surface area for exposure to the alcohol and alkali. For example, using soybeans as the trial feedstock (as shown below), the feedstock was flaked to approx. 0.2-2 mm in thickness and mixed in a sealed glass vessel with an alcohol (e.g., C₁₋₄ alcohol such as methanol, ethanol, isopropanol) and alkali (e.g., NaOH, KOH). The mixture was agitated by swirling during incubation at 60° C. or 23° C. Analysis of the liquid phase after just one hr, the briefest incubation time examined, showed fatty acid methyl esters not only to be present but to be the predominant chemical species. Thus, surprisingly, it is possible to synthesize fatty acid alky esters by direct transesterification of the lipid-linked fatty acids residing in the lipid source (e.g., oilseed). The present process surprisingly allows the production of esters from lipids that are residing in a structurally complex and heterogenous material (for example in an oilseed like soy); the feedstock has not been treated to release the lipid components of the feedstock, for example by the use of conventional organic solvent extraction or extruder-expeller technology well known in the edible oils trade.

Fatty acid alkyl esters may be prepared from the lipid-linked fatty acids (e.g., acylglycerides, phosphoglycerides) in the feedstock by adding an excess (in molar terms) of an alcohol (e.g., lower alkyl alcohols, preferably methanol or ethanol when the product is to be employed as, for example, a diesel engine fuel) and an alkaline catalyst (e.g., KOH, NaOH).

Transesterification will occur in virtually any volume of alcohol/alkali able to wet the ground or flaked lipid source (e.g., oilseed). Larger volumes give more complete transesterification. With regard to the amount of alkali required, when the method is conducted using the reactor geometry and reactants described herein (e.g., flaked soybeans, methanol, and alkali shaken in a sealed container incubated at 60° C.), virtually quantitative amounts of transesterification occur at a sodium hydroxide concentration of about 1.5% (wt. basis, i.e. about 0.37N or about a 67:1 molar ratio of alcohol:alkali) in alcohol. Virtually the same degree of transesterification occurs at lower alkali concentrations (e.g., down to approximately 0.1N), and contaminating free fatty acid levels are reduced. At alkali concentrations of approximately 0.05N and below, transesterification is less efficient and triglycerides are also found in the product mixture.

When conducted using the reactor geometry and reactants described herein, consisting of flaked soybeans, methanol and alkali shaken in a sealed container, generally about 0.04-about 25 ml (e.g., 0.04-25 ml) of alcohol per gram of lipid source (e.g., oilseed) are utilized (preferably about 0.1-about 10 ml (e.g., 0.1-10 ml) of alcohol, more preferably about 0.5-3.0 ml (e.g., 0.5-3.0 ml) of alcohol). This corresponds to molar proportions of alcohol to lipid source (e.g., oilseed) triglyceride of 3.38-2178:1,8.71-871:1, and 43.6-261:1 respectively.

The amounts of alcohol necessary, in the context of reaction stoichiometry, to achieve full esterification are quite small. For example, the volume of methanol theoretically necessary and sufficient to completely transesterify the triglycerides in 1 gm of soybean with an oil content of 25% is only 0.035 ml. Larger volumes are specified above due largely to the fact that the flaked lipid source (e.g., oilseed) substrate matrix both binds and passively retains a certain amount of the alkaline alcohol solution. The disadvantage of the use of substantially larger than stochiometric amounts of alcohol is the effort, difficulty and expense of removing unreacted alcohol from the fatty acid ester product at the end of the reaction.

The specifications above are driven not solely by the chemical requirements of the system, but also by the reaction geometry employed. Variations in reactor design that impact such parameters as the amount of alkaline alcohol required to sufficiently expose all lipid in the lipid source (e.g., oilseed) to that reactant solution will impact the amount of alkaline alcohol that is optimum for maximum ester production. Alternate reaction geometries, such as but not limited to trickle-through extraction devices that pass the alkaline alcohol solution over the lipid source (e.g., oilseed) multiple times, can be imagined that might perform optimally with greater or lesser amounts of alkaline alcohol. The specifications listed above are not intended to eliminate the possibility that such other optima might exist for other reaction geometries.

At a reactant ratio of 5:0.37:0.0055 (gm flaked seed:mole alcohol:mole alkali), the removal of triglyceride from the substrate, in the case of soybeans, is virtually complete (98% as measured by Soxhlet extraction of post-transesterification flakes) within one hour of reaction at 60° C., and fatty acid ester synthesis is substantial.

To prevent evaporation of the alcohol reactant, the reactions are conducted in sealed containers in a preferred embodiment of the invention. No further added pressure need be applied in order to achieve transesterification, though the reaction may proceed at increased pressures. Generally, the method is conducted at atmospheric pressure.

The reaction proceeds well at room temperature (e.g., about 22° C.). Higher reaction temperatures increase the rate of transesterification and yield more fatty acid ester. Above about 65° C., extra containment procedures may be necessary due to elevated pressures. Such higher temperatures and pressures are not necessary to obtain significant amounts of transesterification. As the reaction temperature is reduced toward normal room temperature (22° C.) the amount of free fatty acid liberated during the reaction is reduced.

Generally, about 3-about 10 ml (e.g., 3-10 ml) of alcohol per gram of oilseed are utilized (preferably about 4-about 9 (e.g., 4-9 ml) of alcohol, more preferably about 6-about 7.5 ml (e.g., 6-7.5 ml) of alcohol). Generally, about 0.02-about 0.18 molar (e.g., 0.02-0.18 molar) of alkali in the alcohol (preferably about 0.06-about 0.13 molar (e.g., 0.06-0.13 molar) of alkali are utilized, more preferably about 0.08-about 0.11 molar (e.g., 0.08-0.11 molar) of alkali).

Generally, the reaction time is usually about 2-about 12 hours (e.g., 2-12 hours), preferably about 8-about 9.5 hours (e.g., 8-9.5 hours), more preferably about 7-about 9 hours (e.g., 7-9 hours). Generally, the reaction temperature is usually about 20°-about 70° C. (e.g., 20°-70° C.), preferably about 20°-about 40° C. (e.g., 20°-40° C.), more preferably about 20°-about 30° C. (e.g., 20°-30° C.). Preferably, the reaction time is about 8 hours (e.g., 8 hours) at about 23° C. (e.g, 23° C.) or about 6 hours (e.g., 6 hours) at about 60° C. (e.g., 60° C.).

The fatty acid alkyl ester product will typically contain less than about 1000 mg FFA (free fatty acids)/g fatty acid alkyl esters; the fatty acid alkyl ester product may contain less than about 800 mg FFA/g fatty acid alkyl esters, less than about 400 mg FFA/g fatty acid alkyl esters, less than about 200 mg FFA/g fatty acid alkyl esters, or less than about 50 mg FFA/g fatty acid alkyl esters. As noted above, production of FFA is reduced when the reaction is conducted at lower temperatures or greater ratios of alcohol to alkali. Generally, the fatty acid alkyl ester product will contain less than about 5% weight basis of unreacted triacylglycerols, unreacted diacylglycerides, and unreacted monoacylglycerides, preferably less than about 1% weight basis of unreacted triacylglycerols, unreacted diacylglycerides, and unreacted monoacylglycerides. The identity of the fatty acid alkyl ester product is determined by the identities of the alcohol and the oil source employed in the reaction. Preferably, in the context of a fuel for compression ignition engines, the fatty acid alkyl ester product is fatty acid ethyl esters or more preferably fatty acid methyl esters.

The following examples are intended only to further illustrate the invention and are not intended to limit the scope of the invention as defined by the claims.

### EXAMPLE 1

Soy bean flakes, produced from soybeans that had been treated at 150° F. for about one minute, the item used industrially in the hexane-extraction based recovery of soybean oil, were obtained from a commercial edible oil extraction facility.

Experiment I: 5 g flaked soybeans were added to two approximately 150 ml screw capped glass bottles as reactors. To one glass bottle was added 15 ml of methyl alcohol in which 0.22 gm of sodium hydroxide had been dissolved. To the other was added 15 ml of isopropyl alcohol containing 0.22 gm sodium hydroxide. The bottles were capped and swirled at 60° C., after 2 hours 0.5 ml was removed and frozen at −20° C. for later analysis. After 17.5 hr., the liquid was removed from atop the flaked beans. The content of the 2 and 17.5 h samples were analyzed by thin layer chromatography (TLC) of 10 microliter samples on silica gel with standards (i.e., soybean tri-, di-, and mono-acylglycerols, free fatty acids, and fatty acid methyl esters). The developing solvent was hexane/diethyl ether/acetic acid (80/20/1, v/v/v). After the run, the plate was air dried, sprayed with concentrated sulfuric acid, and heated to display the locations of carbonaceous compounds.

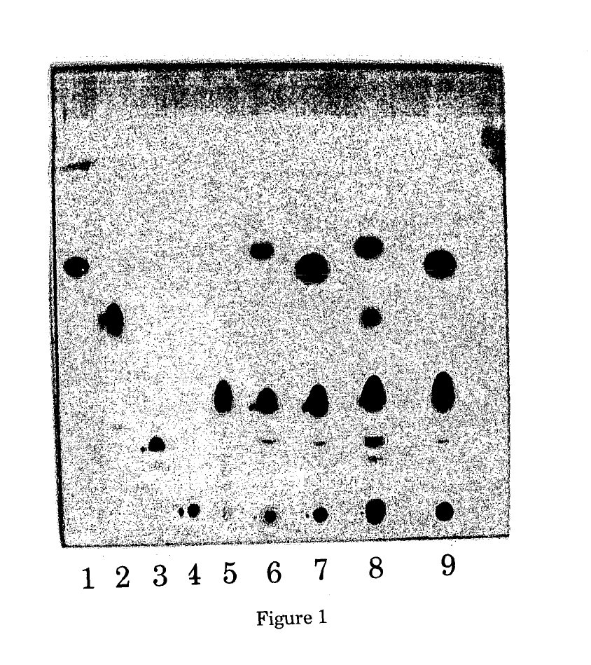

The results are shown in FIG. 1 (counting lanes from the left in the Figure):

Lane 1: standard (known soybean fatty acid methyl ester)

Lane 2: standard (known soybean triglycerides)

Lane 3: standard (1,3-diacylglycerol)

Lane 4: standard (1-monoacylglycerol)

Lane 5: standard (soybean free fatty acids)

Lane 6: transesterification attempt, alcohol=isopropyl, 2 hr. reaction.

Lane 7: transesterification attempt, alcohol=methyl, 2 hr. reaction

Lane 8: transesterification attempt, alcohol=isopropyl, 17.5 hr

Lane 9: transesterification attempt alcohol=methyl, 17.5 hr

Interpretation of FIG. 1: Fatty acid ester was present, predominant in all reactions, in tubes 6-8. Of alcohols tested, methanol was superior to isopropanol in yield of ester. Substantial amounts of free fatty acids were present in all reactions. Except at extended reaction time with isopropanol, no triglyceride was present in the extracts. Diacylglycerol and monoacylglycerol were present in all reactions, lanes 6-8. Thus in situ transesterification was demonstrated to produce fatty acid esters. Ample free fatty acids were also produced which suggested that triglyceride hydrolysis occurred.

Experiment II: The physical setup was the same as Experiment I, except that the bean flakes were dried under vacuum to a constant weight prior to use, in an attempt to remove water that might be the cause of the free fatty acid production noted above. Alcohols tested were methanol, methanol dried with sodium sulfate (which, without being bound by theory, supposedly reduces water content and reduces generation of free fatty acids), and isopropanol. Also conducted was a reaction containing methanol but no sodium hydroxide to test whether the latter was required to achieve esterification. Incubation times: 1 and 16.25 h. Analysis by TLC as in Experiment I.

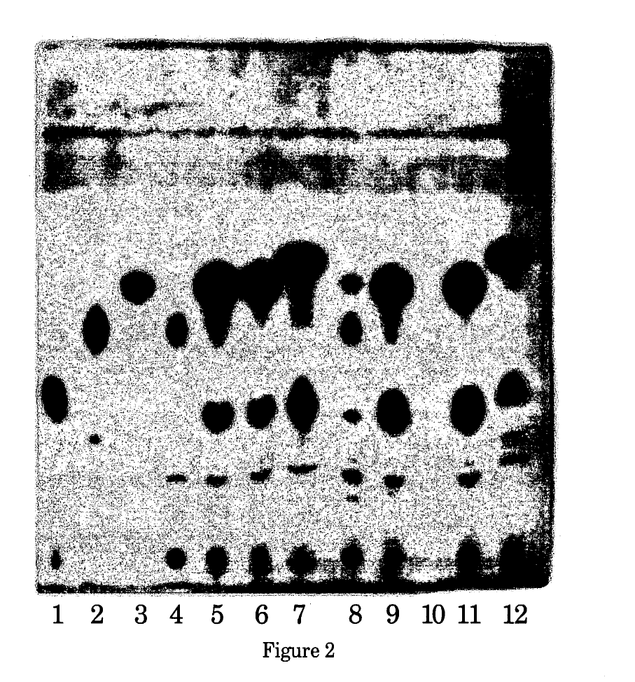

The results are shown in FIG. 2 (counting lanes from the left in the Figure):

Lane 1: standard (soybean free fatty acid)

Lane 2: standard (soybean triglycerides)

Lane 3: standard (soybean fatty acid methyl ester)

Lane 4: methanol, no sodium hydroxide, 1 hr. reaction

Lane 5: methanol plus base, 1 hr.

Lane 6: dry methanol plus base, 1 hr.

Lane 7: isopropanol plus base, 1 hr.

Lane 8: methanol alone, 16.25 hr.

Lane 9: same as 5, 16.25 h.

Lane 10: blank

Lane 11: same as 6, 16.25 hours

Lane 12: same as 7, 16.25 hours

Interpretation of FIG. 2: Fatty acid ester was not made in the absence of sodium hydroxide in 1 hr. Some may have been made over the course of 16.25 hr. reaction. Some triglyceride was extracted by methanol without base. Ester was produced in 1 hr reactions and was predominant species present. The longer reaction time appeared to produce no greater ester yield. Free fatty acid was produced in all reactions containing alcohol and base. Drying the beans and the alcohol did not retard free fatty acid production. Again, both methanol and isopropanol were able to achieve strong ester production. Amount of ester was roughly the same with either.

Experiment III: Physical setup was same as Experiment I. In some tubes dry bean flakes were the substrate, in others the flakes were used as received, again to investigate the theory that water might be the cause of the free fatty acids being produced. Alcohols tested were methanol, ethanol, and ethanol dried with sodium sulfate (which, without being bound by theory, supposedly reduces water content and reduces generation of free fatty acids). All reactions contained sodium hydroxide. Incubation times: 1 and 16.5 h. Analysis by TLC as in Experiment I.

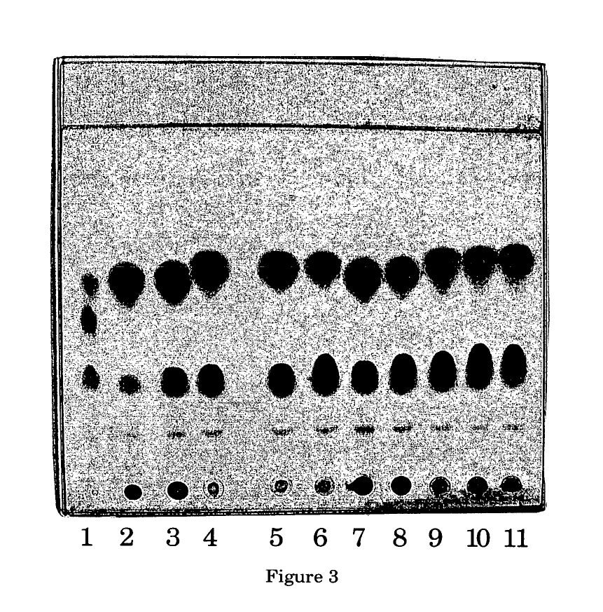

The results are shown in FIG. 3 (counting lanes from the left in the Figure):

Lane 1: standards (mix containing FFA, triglyceride, methyl ester)

Lane 2: methanol, dry flakes, 1 hr.

Lane 3: methanol, flakes as received, 1 hr.

Lane 4: ethanol, dry flakes, 1 hr.

Lane 5: ethanol, flakes as received, 1 hr.

Lane 6: dry ethanol, dry flakes, 1 hr.

Lane 7: same as 2, 16.5 h

Lane 8: same as 3, 16.5 hr.

Lane 9: same as 4, 16.5 h.

Lane 10: same as 5, 16.5 hr.

Lane 11: same as 6, 16.25

Interpretation of FIG. 3: Fatty acid ester was made in substantial amounts under all circumstances (i.e., ethanol also was an acceptable alcohol). One hour incubations were sufficient, 16.5 hr may yield no additional ester product. Predominant production of free fatty acid occurred. Removal of water from reactants reduced this little if at all. As previously seen, minor amounts of di- and perhaps mono-acylglycerols were present.

Experiment IV: The ‘Soxhlet extractor’ is a standard device used to extract solvent-soluble components from a material. It repeatedly passes a batch of solvent over a charge of material, successively extracting a higher and higher proportion of soluble materials. The present experiment investigated the ability of such an approach to achieve transesterification of the fatty acids in soybeans.

The extraction chamber of a Soxhlet extractor was charged with 15 gm of soybean flakes. To the liquid chamber was added 100 mL of ethanol and sodium hydroxide to 1%. Heat was applied to boil the ethanol solution, which was condensed and allowed to drip onto the flakes. Extraction was continued for either 1.75 or 5.5 hr. To examine the effect of trace water content, the reactions were conducted (1) using ethanol and soy flakes as received, and (2) with soy flakes that had been dried by lyophilization and ethanol dried by sodium sulfate pretreatment.

Thin layer chromatography was conducted as above to analyze for formation of ester.

The results are shown in FIG. 3 (counting lanes from the left in the Figure; Note: ethanol throughout):

Lane 1: standards (mix containing, FFA, triglyceride, methyl ester)

Lane 2: 5 uL, dry reaction, 1.75 hr incubation.

Lane 3: 15 uL, dry reaction, 1.75 hr incubation

Lane 4: 5 uL, not dried, 1.75 hr.

Lane 5: 15 uL, not dried, 1.75 hr.

Lane 6: same as #2, but 5.5 hr. incubation

Lane 7: same as #3, but 5.5 hr.

Lane 8: same as #4, but 5.5 hr.

Lane 9: same as #5, but 5.5 hr.

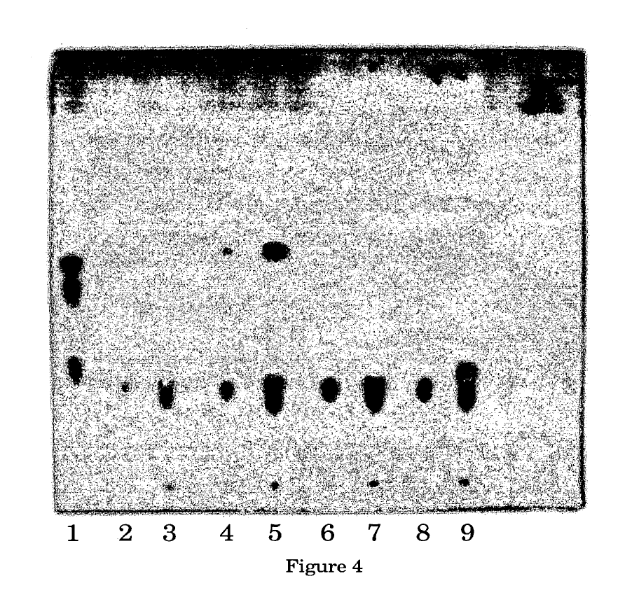

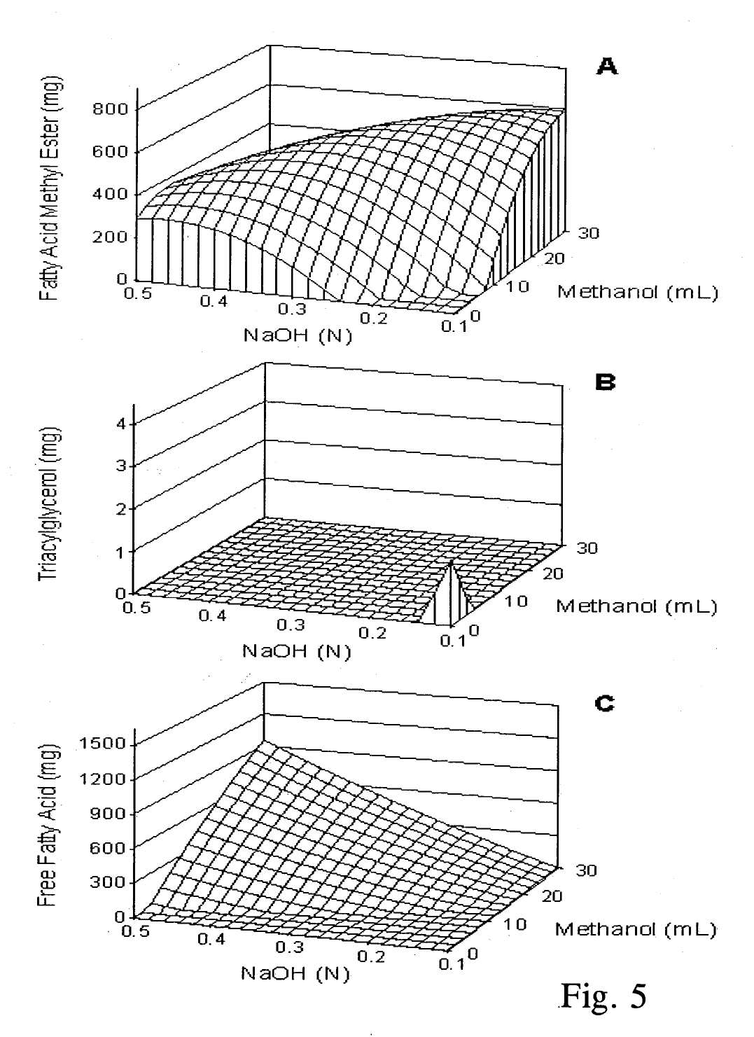

Interpretation of FIG. 4: TLC gave evidence of fatty acid ester production with esters being present after 1.75 hr. incubation. Largest ester spot was obtained in reaction using undried reagents (lane 5). Absence of esters at 5.5 hr suggested destruction of those formed at earlier incubation times during incubations of extended duration, a hypothesis consistent with the free fatty acids present both after 1.75 hr but more so at 5.5 hr. There may have been monoglycerides present in material lingering at origin on TLC plates. It must be born in mind that the liquid phase volume in this reaction was 125 mL, whereas in experiment I-III it was just 15 mL. Thus, the fact that the ester spot was fainter in this experiment may not necessarily mean that transesterification was weaker with the Soxhlet approach; it may just be due to the greater dilution of the ester.

The disclosed method eliminates the costs of oil extraction and refining in the production of fatty acid alkyl esters. These costs constitute roughly 60% of the cost of refined oil, which itself accounts for roughly 75% of the cost of biodiesel production. Thus, it is calculated that elimination of extraction and refining could reduce the cost of biodiesel production by approximately 45%. Production costs are approximately $2.20/gal when the feedstock is refined oil. A 45% decrease in feedstock cost would reduce this overall production cost to $1.21/gal. With annual sales volumes of 20 million gallons, this amounts to a savings of approximately $14 million in production costs if all that biodiesel were originally made from refined oil (as much, but not all, is). It is likely that a method offering such a reduction in cost would be adopted by a large proportion of producers and could be sufficient to assure the economic competitiveness of biodiesel.

Although simple batch methods have been tried to date, the development of a continuous process can be readily envisioned by one skilled in the art.

### EXAMPLE 2

Chemicals: Flaked soybeans, prepared for hexane extraction in a commercial oil plant, had a thickness of 0.28 to 0.35 mm. The oil content of the flakes, determined by extraction with hexane for 4.5 h in a Soxhlet apparatus, was 23.9% (mass basis). Their moisture content, determined by overnight lyophilization, was 7.4% (mass basis). These values are typical for the oil and water contents of commercial flakes soybeans (Williams, M. A., and R. J. Hron, Sr., Obtaining Oils and Fats from Source Materials, in Bailey's Industrial Oil & Fat Products, Fifth Edn., Vol. 4, edited by Y. H. Hui, John Wiley & Sons, Inc. New York, pp. 61-155). Flakes were stored under nitrogen at −20° C.

Lipid standards were obtained from Sigma-Aldrich. Palmitic, stearic, oleic, linoleic, and linolenic acids mixed in amounts proportional to their mass abundance in soybean oil (Fritz, E., and R. W. Johnson, Raw Materials for Fatty Acids, in Fatty Acids in Industry, Processes, Properties, Derivatives, Applications, edited by R. W. Johnson, and E. Fritz, Marcel Dekker, New York, 1989, pp. 1-20.) served as the FFA standard. A mixture of FAME whose composition reflected the fatty acid content of soy oil (RM-1) was the product of Matreya, Inc. (Pleasant Gap, Pa.). Necessary reagents for the determination of glycerol were obtained as components of a triglyceride assay kit (Sigma-Aldrich). Organic solvents were B&J Brand™ High Purity Grade (Burdick & Jackson, Inc., Muskegon, Mich.). Sulfuric acid (96.3%) was the product of Mallinckrodt Baker (Paris, Ky.). Other reagents were Analytical Reagent grade quality or better.

Conduct and optimization of in situ transesterification: Flaked soybeans (5.00 g unless otherwise stated) were mixed with alkaline alcohol (an alcohol, in this case methanol, in which alkali, in this case sodium hydroxide, is dissolved) in screw-capped bottles of capacity at least 5 times the reaction volume. These were mixed by orbital shaking at a speed sufficient to keep the flakes well suspended. Following reaction, bottles were allowed to sit for 15 min at room temperature to allow the flakes to settle and the reaction to cool. The liquid phase was removed and, for qualitative analysis, directly analyzed by TLC. For quantitative analysis the spent flakes were washed twice by resuspension in 10 mL methanol and the washes were pooled with the reaction liquid. The combined methanol layers were centrifuged (15 min 5900×g) and the resulting supernatant removed. Following its dilution to 40 mL with methanol, 1 mL was mixed with 10 mL of 2M KCl-HCl buffer, pH 1.0, and extracted with 10 mL hexane. The organic layer was recovered and its lipid components analyzed by HPLC.

Focusing on the reaction with methanol, Central Composite Response Surface design methods (Box, G.E.P., W. G. Hunter, and J. S. Hunter, Statistics for Experimenters, Wiley, New York 1978) were employed to coordinately investigate the effects and interactions of the amount of alkaline methanol, its NaOH concentration, and reaction time on the yields of FAME, FFA and unreacted acylglycerols (AG) in the liquid phase. Preliminary studies (data not shown) were conducted to focus the statistically designed work in the region of variable space giving the highest FAME production.

Two temperatures were investigated: 60° C. and 23° C. (room temperature). For the 60° C. reaction, the amounts of alkaline methanol tested were 7.5 (the minimum to cover 5 g of flakes), 12.1, 18.7, 25.4 and 30.0 mL; the NaOH concentrations tested were 0.05, 0.14, 0.275, 0.41 and 0.5 N, and reaction times were 0.25, 1.8, 4.00, 6.2 and 7.8 h. For reactions at room temperature, the amounts of alkaline methanol tested were 14.2, 18.7, 25.4, 32.1 and 36.7 mL; NaOH concentrations were 0.02, 0.052, 0.10, 0.148 and 0.18 N; and reaction times were 2.5, 4.0, 6.2, 8.5 and 10.0 h. Each experimental series involved 20 reactions at various combinations of these levels.

FAME, FFA and AG levels were quantitated by HPLC following sample preparation as previously described in this section. Best-fit equations correlating this data with the composition of the reactions were constructed using SAS/STAT software (SAS/STAT User's Guide, Version 8, SAS Institute Inc., Cary, N.C., 1999). Numerical analysis of these equations and examination of the corresponding three dimensional surfaces allowed identification of the conditions predicted to give maximum FAME yield with minimum contaminating FFA and AG.

Determination of transesterification efficiency (room temperature): Samples (100 g, conducted in duplicate) of soy flakes were subjected to in situ transesterification at room temperature under identified optimal reaction conditions (680 mL of 0.1 N NaOH in methanol, 7.75 h incubation). After cooling and settling of the flakes the liquid phase was recovered by filtration. The flakes were washed three times by resuspension in 150 mL methanol for 10 min each, and the washes pooled with the reaction liquid. The extracted flakes were air-dried, lyophilized to dryness, and their mass determined.

To determine the efficiency of lipid removal from the flakes during in situ transesterification, 20.0 g of the dried, post-reaction flakes was extracted for 4 h with 150 mL hexane in a Soxhlet apparatus. The liquid phase was recovered, its hexane removed under vacuum, and the acylglycerol content of the extract was determined by HPLC.

The transesterification reaction liquid phase and the liquid from the post-transesterification washes of the flakes were pooled, adjusted to pH 3 with concentrated HCl, and the methanol removed under vacuum. The resulting syrup was resuspended in 150 mL water and extracted 5 times with 300 mL of hexane. The pooled organic phases were dried over sodium sulfate, recovered, and their hexane removed under vacuum. The mass of the resulting liquid was determined, and its FAME and FFA contents measured by HPLC.

Determination of fate of glycerol: Samples (28-30 g, conducted in duplicate) of the dried post-transesterification flakes generated in the preceding section were washed by swirling for 30 min. each in 2×300 mL water. The washes were recovered by filtration, pooled, adjusted to neutrality with HCl, and the glycerol content was determined.

Glycerol contents of this spent-flake wash, and of the water-soluble portion of the original reaction liquid, prepared as described in the preceding section, were determined by an enzymatic assay linking the glycerol kinase-catalyzed phosphorylation of glycerol, via the intermediate actions of pyruvate kinase and lactate dyhydrogenase, to the oxidation of NADH (Instruction Manual, Triglycerides Determination Kit, Sigma-Aldrich, St. Louis, Procedure No. 320-UA, 1996). Solutions of glycerol of known concentration served as reference standards.

Thin layer chromatography: TLC was performed on 250 μm Silica G plates (Analtech, Newark, Del.). The developing solvent was hexane:diethylether:acetic acid (80:20:1, volume basis). Spots were visualized by spraying with sulfuric acid and charring on a hotplate.

High performance liquid chromatography: The presence and amounts of FAME, FFA and AGs were determined by HPLC on a silica column (Haas, M. J., and K. M. Scott, J. Am. Oil Chem. Soc., 73:1393-1401 (1996)). Peaks were eluted with gradients of isopropanol and water in hexane-0.6% acetic acid (v/v), detected by evaporative light scattering, and quantitated by reference to standard curves constructed with known pure compounds. Minimum detectable levels of lipid species per reaction conducted as described above (“Conduct and optimization of in situ transesterification”) were: FAME: 60 mg; FFA: 1.1 mg; triacylglycerols, diacylglycerols (DAG), monoacylglycerols (MAG): 1.8 μg; phosphoacylglycerols: 2.7 μg.

Results and Discussion:

Preliminary investigations demonstrated that even brief incubations of soy flakes in alkaline solutions of simple alcohols at 60° C. resulted in the production of fatty acid alkyl esters. This occurred with methanol, ethanol and isopropanol, suggesting that the effect was a general one. Under alkaline conditions, ester production with methanol appeared as strong as with less polar alcohols. FFA were produced during alkaline in situ transesterification.

Optimization of reaction: Optimization of reaction conditions has the potential to reduce reagent consumption, increase yields and decrease contamination by FFA and AG. Due to the industrial importance of the methyl esters of fatty acids, we focused on optimizing conditions for in situ transesterification with this alcohol though it is expected that similar results will occur with other alcohols.

Two reaction temperatures were investigated: (1) 60° C., which is sufficiently warm to achieve rapid reaction, yet is below the boiling point of the system, eliminating the need for pressurized equipment, and (2) 23° C. (room temp.), at which heating of the reaction is not required and at which the reduced volatility of the alcohol component eases vapor containment and reduces the need for solvent replacement. Reaction conditions yielding high degrees of transesterification with low levels of FFA and free AG were sought. A low content of FFA is desirable because these represent lost potential FAME. Also, low FFA levels are specified for FAME preparations intended for use as biodiesel (Standard Specification for Biodiesel Fuel (B100) Blend Stock for Distillate Fuels, Designation D 6751-02, American Society for Testing and Materials, West Conshohocken, Pa. (2002)), which necessitates additional cleanup steps for high-FFA preparations.

The best-fit second-order response surfaces to describe the production of FAME, FFA and TAG in reactions conducted at 60° C. are given by Eqn. 1-3:

FAME=−1280+138T+93.7V+6160B−0.464T²−2.89TV−275TB−1.26V²−137VB+6010B² (1)

FFA=−184+34.4T+5.16V+771B−8.22T²+2.02TV+96.2TB−0.347V²+75.8VB+1140B² (2)

TAG=4.62+0.956T+0.253V−54.2B−0.0661T²−0.0117TV−0.587TB−0.00735V²+0.196VB+78.4B² (3)

where FAME, FFA and TAG are expressed as mg/reaction, T=incubation time (hours), V=volume of alkaline alcohol (mL), and B=alkali concentration (Normality) in the alcohol. These equations gave acceptable fits to the experimental data, with R² values of 86.4% for FAME, 97.5% for FFA, and 64.0% for TAG. Di-, mono- and phospho-AGs were not detected in FAME samples prepared at 60° C.

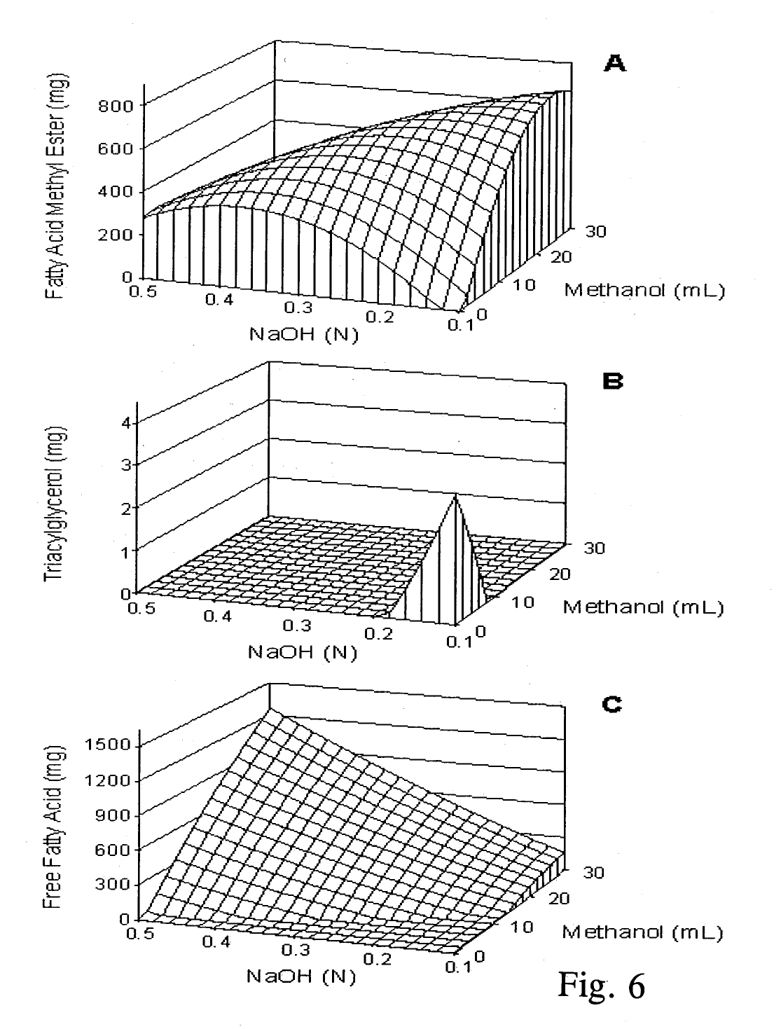

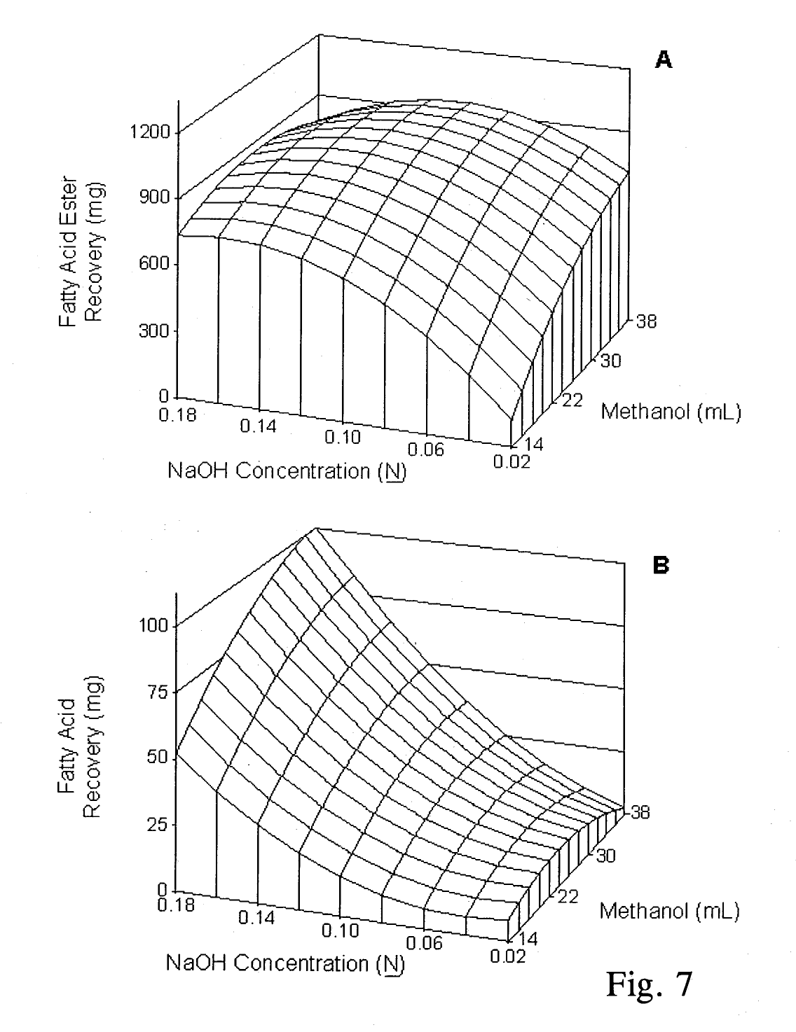

Eqn. 1-3 allowed construction of surfaces describing the levels of TAG, FAME, and FFA in the reaction liquid as a function of its composition during in situ transesterification at 60° C. (FIGS. 6 and 7). After two hours, FAME production was nearly complete; additional incubation, to 6 hr total, only slightly increased the yield. In fact, transesterification proceeded rapidly, with some reactions producing 80% of the FAME yield seen at 6 h. within 15 min. Incubation beyond 6 h did not further increase yield. The level of unreacted oilseed TAG, extracted from the seeds but not transesterified, was low over virtually the entire coordinate space examined (FIGS. 6 and 7). FFA levels were also low in reactions containing low alkali concentrations and low to moderate amounts of alcohol (FIGS. 6 and 7). Numerical optimization, and examination of FIGS. 6 and 7, indicated that at 60° C. the conditions resulting in high FAME production with low contamination by FFA and TAG from 5 gm soy flakes were 12 to 25 mL methanol, an NaOH concentration between 0.1 and 0.2 N, and a reaction time of approximately 6 h. The greater the alcohol volume the lower the alkali concentration required to give good yields of FAME with low FFA and AG contamination. Using 22.5 mL of 0.1 N NaOH the predicted amounts of FAME, FFA and TAG were 762, 62 and 3 mg, respectively. Upon reducing the methanol to 12.5 mL and increasing NaOH to 0.18 N the predicted product composition after 7.7 h reaction was 675 mg FAME, less than 1 mg FFA, and no TAG. These latter conditions correspond to a molar ratio of 226:1:1.6 for methanol:TAG:alkali. By comparison, optimal conditions for the conventional alkali-catalyzed transesterification of refined soy oil at 60° C. are molar ratios of 6:1:0.22 for methanol:TAG:NaOH (11). Thus, in the present configuration the in situ method employs about 38 times more alcohol and 7 times more alkali than does the conventional method. The excess reagents could be recovered for reuse if desired.

When conducted at room temperature, no tri-, di-, mono- or phospho-AGs were detected in the liquid phase following transesterification. The best-fit second-order response surfaces to describe the FAME and FFA levels as a function of the composition of the reaction were:

FAME=−1355+129.2T+63.22V+13710B−8.214T²−0.2204TV−147.0TB−0.7243V²−143.8VB−33360B² (4)

FFA=−21.78−9.141T+2.050V−733.0B−1.005T²+0.0570TV+41.72TB−0.0580V²+14.22VB+2393B² (5)

The R² values for the fits of these equations to the data were 93.7% for FAME and 98.6% for FFA, indicating that the data were well modeled by the equations.

Using Eqn. 4 and 5, predictive surfaces were constructed to describe the composition of the reaction products as a function of alkali concentration, amount of methanol, and reaction time at room temperature (FIG. 8). Maximum FAME production was achieved after about 8 h of reaction, with 90% of maximum occurring by 2 h (data not shown). For reactions of about 8 h duration, the best yields of FAME and lowest levels of contamination by FFA were predicted for reactions containing 5 gm flakes and 30 mL or more of methanol (minimum molar ratio of methanol:triglyceride=543) with an NaOH concentration of 0.09 N (molar ratio of NaOH:triglyceride=2.0). Predicted FAME and FFA levels under these conditions were on the order of 940 and 35 mg, respectively. This is a higher FAME yield and lower FFA level than predicted for reactions under optimal conditions at 60° C. As at 60° C., the molar reagent requirements at room temperature are substantially greater than those for alkaline transesterification of refined oil (11): 90 times more methanol and 9 times more NaOH. The methanol requirement at room temperature was also approximately 2.4 times that at 60° C. (above), but the additional expense of this increase may be compensated for by the reduced costs of room temperature operation.

Transesterification efficiency: The FAME fraction recovered after in situ transesterification of 100 g of soy flakes at room temperature for 7.75 h under optimal conditions (680 mL of 0.1N NaOH in methanol) weighed 19.5 g and was determined by HPLC to contain 18.9 g (97 wt %) FAME and 0.14 g (0.72 wt %) FFA (all data are means of replicate reactions; individual values differed from the means by no more than 4%). Given an initial lipid content of 23.9% in the flakes, the theoretical maximum FAME recovery was 23.8 gm. Overall FAME recovery was thus 79.4% of theoretical. No acylglycerols were detected in the FAME product.

Soy flakes lost 31.9% of their mass during in situ transesterification at room temperature. This exceeds the total lipid content of the flakes (23.9%) but is consistent with a high degree of removal of water (original content: 7.4%) as well as lipid during transesterification. Hexane extraction of dried post-transesterification flakes removed 1.3 g of material. HPLC analysis indicated that triacylglycerols made up 83% of this material. Thus, approximately 1.1 g, 5% of the lipid content of the flakes, was neither extracted nor transesterified during the in situ reaction. This would contribute to the less than quantitative recovery of FAME that was observed.

Using acidic methanol under reflux, Kildiran et al. (J. Am. Oil Chem. Soc., 73: 225-228 (1996)) observed a maximum extraction of 40% of the oil from finely ground soy beans, with only 55% transesterification of this extracted oil, giving an overall FAME yield of 22%. As opposed to acid catalysis, the alkaline room temperature reaction conducted here surprisingly achieved a much greater removal of oil from the substrate (95%) and more effective transesterification of the extracted oil (84%). Some of the unrecovered lipid and ester may have been lost to a small emulsion layer that formed during extraction of the samples with water and hexane during analysis.

Fate of glycerol: Glycerol is a coproduct of the transesterification process. There was interest in determining the fate of glycerol in the in situ process, since current biodiesel specifications (Standard Specification for Biodiesel Fuel (B100) Blend Stock for Distillate Fuels, Designation D 6751-02, American Society for Testing and Materials, West Conshohocken, Pa. (2002)) limit the amount allowed, and since its recovery could give rise to another product stream. Also, since a typical use of solvent-extracted oilseed flakes is as an animal feed, there was interest in determining the degree to which glycerol might be bound to the flake fraction, where it might affect nutritional performance of the flakes.

Aqueous extraction was used to recover glycerol from the FAME and the spent-flake fractions of the 100 g reactions described above. Enzymatic assay determined that recovered glycerol was located predominantly (93%) in the liquid fraction following transesterification: its contents in the FAME and spent-flake fractions were 1.9 and 0.14 g, respectively. The sum of these values accounts for approximately 84% of maximum theoretical glycerol recovery. Some of the remainder can be attributed to the 5% of the oil fraction that was not extracted from the flakes.

Overview: As demonstrated previously (Kildiran, G., S. et al., J. Am. Oil Chem. Soc., 73: 225-228 (1996)) methanol itself is a poor vegetable oil extractant. We detected only negligible amounts of ester following a 4 h extraction of soy flakes with methanolic NaOH in a Soxhlet extractor. Presumably this is because the flake bed is exposed only to the methanol component under Soxhlet conditions. As shown here, however, incubation of soy flakes with alkaline methanol surprisingly results in the recovery of substantial amounts of fatty acid ester.

Our alkali-catalyzed process offers the advantages of (a) efficient operation using soy flakes prepared by current industrial technology rather than requiring completely pulverized beans, (b) use of less reagents and milder reaction conditions, and more importantly, (c) substantially higher ester yields. Although we have investigated only soy flakes as a substrate here, we fully expect that the technique will be applicable to other oilseeds as well. In addition, this process should lend itself to continuous operation, a desirable format that also may increase ester yields.

All of the references cited herein are incorporated by reference in their entirety. Also incorporated by reference in their entirety are the following references: Freedman, B., et al., J. Am. Oil Chem. Soc., 61(10):1638-1643 (1984); Haas, M. J., et al., J. Am. Oil Chem. Soc., 77:373-379 (2000); Kildiran, G., S. et al., J. Am. Oil Chem. Soc., 73: 225-228 (1996). U.S. patent application Ser. No. 09/400,799, filed on Sep. 22, 1999, is incorporated by reference in its entirety. U.S. Provisional Patent Application Serial No. 60/347,163, filed on Jan. 9, 2002, is incorporated by reference in its entirety; U.S. patent application Ser. No. 10/337,604, filed on Jan. 7, 2003, is incorporated by reference in its entirety

Thus, in view of the above, the present invention concerns (in part) the following:

A method for producing fatty acid alkyl esters, comprising (consisting essentially of or consisting of) transesterifying a feedstock containing lipid-linked fatty acids with an alcohol and an alkaline catalyst to form fatty acid alkyl esters, wherein said feedstock is selected from the group consisting of soy, coconut, corn, cotton, flax, palm, rapeseed/canola, safflower, sunflower, animal fats and oil, and mixtures thereof, and wherein said feedstock has not been treated to release the lipid components of said feedstock.

The above method wherein the feedstock is soy.

The above method, wherein the feedstock is coconut or palm.

The above method, wherein the feedstock is rapeseed/canola.

The above method, wherein the fatty acid alkyl esters are fatty acid methyl esters or fatty acid ethyl esters.

The above method, wherein the alcohol is a C₁₋₄ alcohol.

The above method, wherein the alcohol is methanol, ethanol, isopropanol, or mixtures thereof.

The above method, wherein the alcohol is methanol, ethanol, or mixtures thereof.

The above method, wherein the alcohol is ethanol.

The above method, wherein the alcohol is methanol.

The above method, wherein the alkali is NaOH, KOH, or mixtures thereof.

The above method, wherein the alkali is NaOH.

The above method, wherein the molar ratio of the alcohol: the alkaline catalyst is about ≦500:1 (e.g., about 67:1).

The above method, wherein the concentration of the alkaline catalyst is about ≧0.05N (e.g., about 0.1N).

The above method, wherein the method utilizes about 0.04-about 25 ml of the alcohol per gram of the feedstock.

The above method, wherein the method utilizes a molar ratio of the alcohol: the feedstock glyceride content of 3.38-2178:1.

The above method, wherein the method utilizes about 3-about 10 ml of said alcohol per gram of the feedstock (or about 4-about 9 or about 6-about 7.5).

The above method, wherein the method utilizes about 0.02-about 0.18 molar of alkali in the alcohol (or about 0.06-about 0.13 molar or about 0.08-about 0.11 molar).

The above method, wherein the reaction time of the method is about 2-about 12 hours (or about 8-about 9.5 hours or about 7-about 9 hours).

The above method, wherein the method is conducted at a reaction temperature of about 20°-about 70° C. (or about 20°-about 40° C. or about 20°-about 30° C.). Preferably, the reaction time is about 8 hours (e.g., 8 hours) at about 23° C. (e.g, 23° C.) or about 6 hours (e.g., 6 hours) at about 60° C. (e.g., 60° C.).

The above method, wherein the fatty acid alkyl esters contain less than about 1000 mg free fatty acids)/g fatty acid alkyl esters (or less than about 800 mg FFA/g fatty acid alkyl esters or less than about 400 mg FFA/g fatty acid alkyl esters or less than about 200 mg FFA/g fatty acid alkyl esters or less than about 50 mg FFA/g fatty acid alkyl esters).

The above method, wherein the fatty acid alkyl esters contain less than about 5% weight basis of unreacted triacylglycerols, unreacted diacylglycerides, and unreacted monoacylglycerides (or less than about 1% weight basis of unreacted triacylglycerols, unreacted diacylglycerides, and unreacted monoacylglycerides).

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

We claim:

1. A method for producing fatty acid alkyl esters, comprising transesterifying a feedstock containing lipid-linked fatty acids with an alcohol and an alkaline catalyst to form fatty acid alkyl esters, wherein said feedstock is selected from the group consisting of soy, coconut, corn, cotton, flax, palm, rapeseed/canola, safflower, sunflower, animal fats and oil, and mixtures thereof, and wherein said feedstock has not been treated to release the lipid components of said feedstock.

2. The method according to claim 1, wherein said feedstock is soy.

3. The method according to claim 1, wherein said feedstock is coconut or palm.

4. The method according to claim 1, wherein said feedstock is rapeseed/canola.

5. The method according to claim 1, wherein said fatty acid alkyl esters are fatty acid methyl esters or fatty acid ethyl esters.

6. The method according to claim 1, wherein said alcohol is a C₁₋₄ alcohol.

7. The method according to claim 1, wherein said alcohol is selected from the group consisting of methanol, ethanol, isopropanol, and mixtures thereof.

8. The method according to claim 1, wherein said alcohol is selected from the group consisting of methanol, ethanol, and mixtures thereof.

9. The method according to claim 1, wherein said alcohol is ethanol.

10. The method according to claim 1, wherein said alcohol is methanol.

11. The method according to claim 1, wherein said alkali is selected from the group consisting of NaOH, KOH, or mixtures thereof.

12. The method according to claim 1, wherein said alkali is NaOH.

13. The method according to claim 1, wherein the molar ratio of said alcohol: said alkaline catalyst is about ≦500:1.

14. The method according to claim 1, wherein the concentration of said alkaline catalyst is about ≧0.05N.

15. The method according to claim 1, wherein said method utilizes about 0.04-about 25 ml of said alcohol per gram of said feedstock.

16. The method according to claim 1, wherein said method utilizes a molar ratio of said alcohol: said feedstock glyceride content of 3.38-2178:1.

17. The method according to claim 1, wherein said method utilizes about 3-about 10 ml of said alcohol per gram of said feedstock.

18. The method according to claim 1, wherein said method utilizes about 0.02-about 0.18 molar of alkali in said alcohol.

19. The method according to claim 1, wherein the reaction time of said method is about 2-about 12 hours.

20. The method according to claim 1, wherein said method is conducted at a reaction temperature of about 20°-about 70° C.

21. The method according to claim 1, wherein said fatty acid alkyl esters contain less than about 1000 mg free fatty acids/g fatty acid alkyl esters.

22. The method according to claim 1, wherein said fatty acid alkyl esters contain less than about 5% weight basis of unreacted triacylglycerols, unreacted diacylglycerides, and unreacted monoacylglycerides.
